(19) United States
(12) Patent Application Publication
(10) Pub. No.: US 2025/0041133 A1
(43) Pub. Date: Feb. 6, 2025

(54) BODY-CONFORMABLE ABSORBENT ARTICLE

(71) Applicant: The Procter & Gamble Company, Cincinnati, OH (US)

(72) Inventors: John Daniel ALGERS, Montgomery, OH (US); John Brian STRUBE, Okeana, OH (US); Connie Michelle DUNAWAY, West Chester, OH (US); Andrew Paul RAPACH, Fairfield, OH (US); Scott Kendyl STANLEY, Mason, OH (US)

(21) Appl. No.: 18/921,307

(22) Filed: Oct. 21, 2024

Related U.S. Application Data
(63) Continuation of application No. PCT/US2023/019015, filed on Apr. 19, 2023.
(60) Provisional application No. 63/334,041, filed on Apr. 22, 2022.

(51) Int. Cl. A61F 13/538

(52) U.S. Cl. CPC A61F 13/538; A61F 2013/5386

(57) ABSTRACT

This invention relates to features of an absorbent article designed to be worn by men for containing and absorbing discharges resulting from urinary incontinence, where the absorbent article is thin and conforms to the male anatomy.

### CROSS REFERENCE TO RELATED APPLICATION

This application is a continuation of, and claims priority under 35 U.S.C. § 120 to, PCT/US2023/019015 filed on Apr. 19, 2023, which claims the benefit, under 35 U.S.C. § 119 (c), of U.S. Provisional Patent Application Ser. No. 63/334,041 filed on Apr. 22, 2022, the entire disclosures of all of which are fully incorporated by reference herein.

### FIELD OF THE INVENTION

This invention relates to features of an absorbent article designed to be worn by men for containing and absorbing discharges resulting from urinary incontinence, where the absorbent article is thin and conforms to the male anatomy.

### BACKGROUND OF THE INVENTION

Absorbent articles, such as incontinence guards, baby diapers, and sanitary napkins, are well known. Absorbent articles are designed to absorb, distribute, and store various types of body exudates, while providing a comfortable, secure fit during use. Absorbent articles are also designed to minimize leakage and rewet of the skin. Absorbent articles differ depending on the target wearer and use. For example, feminine sanitary napkins are designed primarily to absorb menses and to be worn adjacent to a woman's pudendum.

Feminine sanitary napkins as well as incontinence pads for women have been known to be used as incontinence products for men. However, such sanitary napkins and incontinence pads for women are not suitable for men suffering from urinary incontinence. Sometimes, though, men suffering from incontinence may choose to use sanitary napkins and incontinence pads for women, because these products are present in their households. Moreover, many men suffer from light incontinence, as opposed to heavy incontinence, and male products designed for heavy incontinence, such as diapers, may be both off-putting and unsuitable.

Light incontinence is common among aging men and may prevent men from leading full and active lives. One group of men who may suffer from light incontinence are men having prostate-related problems. For example, following surgery to correct certain prostate-related problems, men may experience drip incontinence. Men suffering from light incontinence may be embarrassed about the condition and may choose to use female products present in their households to maintain discretion (and avoid having to shop for a men's incontinence product). Men suffering from light incontinence, however, experience important differences from both menstruating women and incontinent women and the use of commercially available feminine care or female incontinence products may not satisfy their specific needs. For example, a problem presented in designing an absorbent article to absorb and contain small, incontinent discharges of urine by a man is the wide variability of penis orientations during an unintended discharge.

Incontinence guards designed specifically for men and intended for light incontinence are known. Existing products, however, do not meet all the needs of a man suffering from light incontinence. Known light incontinence products for men include products that surround the genitals of the male wearer, like an athletic cup. These products may have several disadvantages, such as excessive warmth near the genitals and stiffness that can result in chafing. Light incontinence pads or guards for men are also known. Known light incontinence guards are designed to be worn inside a user's underwear and to provide protection against light urine leakage. Known light incontinence guards are generally provided with adhesives to allow the guards to be attached to wearers' underwear. Incontinence guards comprising fluff pulp are known, but these guards tend to be thick and more noticeable for the user. Light incontinence guards comprising a web-based material are known, and these guards tend to be relatively thin. However, such thin, web-based light incontinence products have tradeoffs, as reducing the thickness of the product may, in fact, increase the stiffness of the product, whereby the product is not able to conform to the male anatomy and is more noticeable to the wearer. Reducing the thickness of the product may also result in insufficient absorption capacity.

There is a need for a light incontinence absorbent article designed for men that is thin, highly flexible, comfortable, and conforms to the male anatomy during use. There is a need for a light incontinence product for men that feels like a user's underwear, is unnoticeable to the user, and is invisible to others under the user's clothing, while providing sufficient absorption capacity and leak protection. Finally, there is a need for a light incontinence product for men that is easy to properly position in the user's underwear and stays in place, even when the user adjusts or shifts his underwear, e.g., to use the restroom. It has surprisingly been found that a light incontinence product designed for the male anatomy and having a selected conformability and thickness provides an optimal wear experience for men suffering from light incontinence.

### SUMMARY OF THE INVENTION

The present disclosure relates to an absorbent article comprising: a. a liquid permeable topsheet; b. a liquid impermeable backsheet; and c. a web-based absorbent core disposed between the topsheet and the backsheet, the absorbent core comprising a fibrous nonwoven web, a cellulose fiber, or a mixture thereof, wherein the absorbent core comprises deformations; wherein the absorbent article exhibits a Conformability Force at 5 mm less than about 3500 N/m and greater than about 260 N/m, and an average dry caliper of about 2 mm to 7 mm.

The present disclosure also relates to an absorbent article comprising: a. a liquid permeable topsheet; b. a liquid impermeable backsheet; and c. a web-based absorbent core disposed between the topsheet and the backsheet, the absorbent core comprising a fibrous nonwoven web, a cellulose fiber, or a mixture thereof, where the absorbent core comprises deformations; where the absorbent article has a tapered, preferably triangular, shape having a wider forward end region that tapers to a narrower rearward end region.

### BRIEF DESCRIPTION OF THE DRAWINGS

FIG. 1 is a plan view of an example of an absorbent article for male incontinence, wearer-facing side up, showing a pattern of deformations on the wearer-facing side.

FIG. 2 is a schematic lateral cross section of the article shown in FIG. 1, taken through line 2-2 in FIG. 1, showing how the layers are stacked.

FIG. 3A-3C is a plan view of another example of an absorbent article for male incontinence, wearer-facing side up, showing a pattern of discrete deformations.

FIGS. 4A-4B are plan views of additional examples of absorbent articles for male incontinence, wearer-facing sides up, showing various patterns of continuous deformations.

FIG. 5 is a CT scan (along a z-direction plane) of a longitudinal cross section of an absorbent article for male incontinence, where the article is rolled from forward end to the rearward end.

FIG. 6 is a simplified schematic side-view depiction of components arranged for a process for deforming a web material.

FIG. 7 is a perspective view of an example of a pair of deforming rollers (“ring rollers”) used to create continuous deformations in a web material or composite web.

FIG. 8 is a view of engaging features of portions of an example of a pair of deforming rollers.

FIG. 9 is a closer view of the features shown in FIG. 8, shown acting upon a web.

FIG. 10 is a schematic cross section view (along a z-direction plane) of an example of a deformed web.

FIG. 11 is a perspective view of an apparatus comprising a pair of deforming rollers used to create discrete deformations in a web material or composition web.

FIG. 12 is a top perspective view of a portion of a deformed, three-dimensional web created using the deforming rollers shown in FIG. 11.

FIG. 13 is a top view of the portion of the deformed, three-dimensional web of FIG. 12.

FIG. 14 is a schematic depiction of equipment used in the Conformability Force Measurement Method, described herein.

### DETAILED DESCRIPTION OF THE INVENTION

“Absorbent article” means a layered product including an absorbent core and configured to be worn externally about the lower torso and/or upper crotch region of a human being, and configured to contain and/or absorb bodily exudates which may include urine, menstrual fluid or feces. Examples of absorbent articles include feminine hygiene pads (also known as catamenial pads or sanitary napkins), male incontinence guards, disposable incontinence pads, absorbent underwear (configured for, e.g., managing incontinence) and diapers.

“Lateral”—with respect to an absorbent article or a component thereof, refers to a direction parallel to a horizontal line tangent to the front surfaces of the upper portions of wearer's legs proximate the torso, when the article is being worn normally and the wearer has assumed an even, square, normal standing position. A “width” dimension of any component or feature of an absorbent article is measured along the lateral direction. When the absorbent article or component thereof is laid out flat on a horizontal surface, the “lateral” direction corresponds with the lateral direction relative the structure when it is worn, as defined above. With respect to an absorbent article that is opened and laid out flat on a horizontal planar surface, “lateral” refers to a direction perpendicular to the longitudinal direction and parallel to the horizontal planar surface.

The “lateral axis” of an absorbent article or component thereof is a lateral line lying in an x-y plane and equally dividing the length of the article or the component when it is opened and laid out flat on a horizontal surface. A lateral axis is perpendicular to a longitudinal axis. “Longitudinal”—with respect to an absorbent article or a component thereof, refers to a direction perpendicular to the lateral direction. A “length” dimension of any component or feature of a layered absorbent article is measured along the longitudinal direction from its forward extent to its rearward extent. When the absorbent article or component thereof is laid out flat on a horizontal surface, the “longitudinal” direction is perpendicular to the lateral direction relative the structure when it is worn, as defined above.

The “longitudinal axis” of an absorbent article or component thereof is a longitudinal line lying in an x-y plane and equally dividing the width of the article, when the article is opened and laid out flat on a horizontal surface. A longitudinal axis is perpendicular to a lateral axis.

“Liquid impermeable”—refers to one or more properties or features of a film, web material or laminate thereof that cause(s) it to resist passage of aqueous liquid therethrough (from one major surface through to the other opposite major surface), under ordinary conditions of use of absorbent articles. A film, web material or laminate thereof may be liquid impermeable, but also vapor permeable (“breathable”).

“Machine direction”—with respect to a process for manufacturing a web material or a laminate or layered arrangement of web materials, refers to the primary direction of conveyance of the materials along the manufacturing line, viewed from above the manufacturing line. It will be understood that the machine direction may change in absolute directional orientation in space at particular locations along the line, if the manufacturing line is so configured. With respect to an individual roller or nip between a pair of rollers, over or in which a web material or combination of web materials is conveyed, laminated or deformed on a manufacturing line, the “machine direction” is ordinarily perpendicular to the axis(es) of the roller(s), and the “cross direction” is ordinarily parallel to the axis(es) of the rollers.

“Permanently mechanically deformed”—means plastically deformed or having individual fiber constituents that have been plastically deformed and/or directionally realigned or reoriented, by application of mechanical force.

“x-y plane,” with reference to an absorbent article or component thereof when laid out flat on a horizontal surface, means any horizontal plane occupied by the horizontal surface or any layer of the article or component.

“z-direction,” with reference to an absorbent article or component thereof when laid out flat on a horizontal surface, is a direction orthogonal to the x-y plane. When the article is being worn by a user (and thus has been urged into a curving configuration), the “z-direction” at any particular point location on the absorbent article refers to a direction normal to the wearer-facing surface of the absorbent article at the particular point location.

The term “discrete deformations,” as used herein, refers to deformations that are discontinuous and separated from each other in all directions parallel to the first plane.

The term “substantially continuous deformations,” as used herein, refers to deformations within which one can connect any two points by an uninterrupted line running entirely within that deformation throughout the line's length. That is, the substantially continuous deformation has a substantial “continuity” in all directions parallel to the first plane and is terminated only at edges of that deformation. The term “substantially,” in conjunction with continuous, is intended to indicate that while an absolute continuity is desired, minor deviations from the absolute continuity may be tolerable as long as those deviations do not appreciably affect the performance of the absorbent article or component thereof, as designed and intended.

With respect to an absorbent article or component thereof, the terms “front,” “rear,” “forward” and “rearward” and similar relative locational terms relate to features or regions of the absorbent article corresponding to the position they would occupy as ordinarily worn by a user, corresponding with the front (anterior) and rear (posterior) of the wearer/user's body when standing.

With respect to an absorbent article, “wearer-facing” is a relative locational term referring to a feature of a component or structure of the article that when in use that lies closer to the wearer than another feature of the component or structure that lies along the same z-direction line. For example, a topsheet has a wearer-facing surface that lies closer to the wearer than the opposite, outward-facing surface of the topsheet.

With respect to an absorbent article, “outward-facing” is a relative locational term referring to a feature of a component or structure of the article that when in use that lies farther from the wearer than another feature of the component or structure that lies along the same z-direction line. For example, a topsheet has an outward-facing surface that lies farther from the wearer than the opposite, wearer-facing surface of the topsheet.

The terms “top,” “bottom,” “upper,” “lower,” “over,” “under,” “beneath,” “superadjacent,” “subjacent,” and similar vertical positional terms, when used herein to refer to layers, components or other features of a wearable absorbent article, are to be interpreted with respect to the article as it would appear when opened and laid out flat on a horizontal surface, with its wearer-facing surface facing upward and outward-facing surface facing downward.

As noted above, a problem presented in designing an absorbent article to be used to contain and absorb unintended (incontinent) small discharges of urine and/or other bodily fluids by a man is the wide variability of penis orientations during an unintended discharge. If an absorbent article does not reliably hold absorbent materials against the wearer's skin to intercept flow from a variety of penis orientations, leakage can result. However, it is also preferable that an absorbent article does not wrap around the male anatomy, as this may cause discomfort during wear.

An absorbent article for male incontinence that is comfortable and provides an optimal wear experience, while reliably protecting against leakage through a variety of body movements and penis positions and over a reasonable duration of wear/use, has not been developed to date. Currently marketed male incontinence guards comprising web-based absorbent cores may be thin, but these products tend to be stiff and less conformable than thicker, fluff pulp-based products. The human body, particularly the male anatomy, has geometrically non-ruled contoured surfaces. Absorbent articles that are stiff will not effectively conform to the male anatomy through ordinary body positions and movements. More conformable, thicker, fluff pulp-based products, however, are more noticeable to the user and may remind the user about his incontinence. The conformability and thickness of an absorbent article for male incontinence may be selected to increase in wear comfort, even through a variety of body movements and penis positions, while providing sufficient absorption capacity and leak protection.

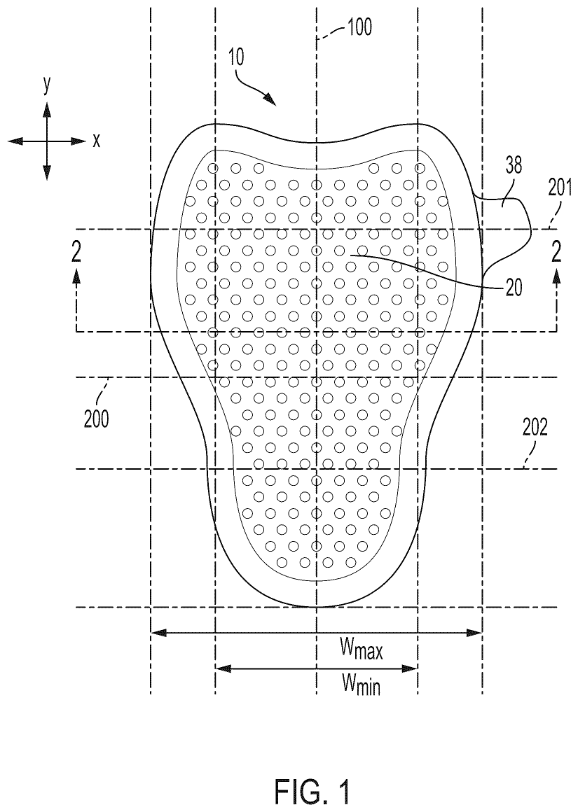

The male incontinence absorbent article 10 of the present disclosure may comprise any suitable shape including but not limited to an oval, a discorectangle, a rectangle, an asymmetric shape, and an hourglass. The absorbent article may be shaped and configured to cover the point where urine is most likely to first contact the absorbent article 10 (the “pee point”). The male pee point is the urethral opening at the end of the penis. However, the male pec point may vary considerably from user to user, due to variation in the orientation or length of the penis. Also, for an individual user, the pec point may vary considerably based on the condition of the penis, which may swell or shrink based on the user's body temperature, or movement of the penis. The male incontinence absorbent article (10) may have a shape that is tapered and generally triangular, as shown in FIG. 1. The absorbent article 10 may comprise a tapered shape having a wider portion in one end region of the article that tapers to a narrower end region in the other end region of the article, preferably where the rearward end region is narrower, while the forward end region is wider.

As shown in FIG. 1, the absorbent article (10) may have a longitudinal axis (100), a lateral axis (200), forward end region lateral axis (201), and a rearward end region lateral axis (202). The longitudinal axis (100) may extend from a forward end edge (80) to a rearward end edge (85) of the absorbent article (10) and may divide the absorbent article (10) into two halves, preferably two substantially symmetrical halves, relative to the longitudinal axis (100), when the absorbent article (10) is opened and laid out flat on a horizontal surface with its wearer-facing surface facing upward and outward-facing surface facing downward, as shown in FIG. 1. The lateral axis (200) may equally divide the overall length L of the absorbent article (10).

The forward end region lateral axis (201) may extend from a leftmost edge (70) to a rightmost edge (75) of the absorbent article (10), when viewing the article (10) with release paper (38) removed, and may divide the absorbent article (10) into a forward end region (60) and a rearward end region (65). The forward end region lateral axis (201) may define the maximum width Wₘₐₓ of the male incontinence absorbent article (10), where the maximum width Wₘₐₓ is the distance from the leftmost edge (70) to the rightmost edge (75), as shown in FIG. 1. The width of the article may taper, continuously or discontinuously, along the longitudinal axis (100) from the forward end region lateral axis (201) to the rearward edge (85) of the article. The width of the article may taper, continuously or discontinuously, along the longitudinal axis (100) from the forward end region lateral axis (201) to the rearward end region lateral axis (202), which is drawn along the minimum width Wₘᵢₙ dimension measured between the side edges (105a, 105b) of the article (10), as shown in FIG. 1.

The leftmost edge (70) and the rightmost edge (75) may each be rounded or pointed (not shown). Preferably, as shown in FIG. 1, the leftmost edge (70) and the rightmost edge (75) may each be rounded or substantially rounded to better conform to the non-ruled, curved surfaces of the user. The forward edge (80) of the article (10) may be linear or curved. Preferably, as shown in FIG. 1, the forward edge (80) is curved to better conform to the curved surfaces of the user. As shown in FIG. 1, the forward edge (80) curves slightly toward the rearward edge (85) of the article (10) near the longitudinal axis (100).

During use, the forward end region (60) is generally placed over the lower torso region of the user and the rearward end region (65) is generally placed over the upper crotch region of the user. The placement need not be exact, provided that the absorbent article (10) covers the male anatomy, particularly the penis.

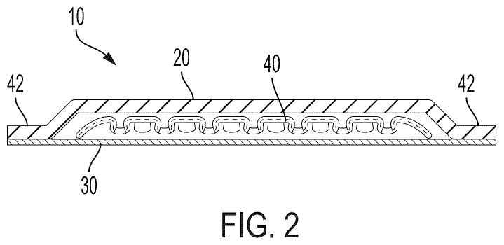

Referring to FIG. 2, a male incontinence absorbent article (10) may include a liquid permeable topshect (20), a liquid impermeable backsheet (30) and an absorbent core (40) disposed between the topsheet (20) and the backsheet (30). The topsheet (20) and the backsheet (30) may extend beyond the outer perimeter of the absorbent core and may be bonded together in laminate configuration to form a perimeter seal (42) by any suitable mechanism, including but not limited to adhesive bonding, thermal bonding, pressure bonding, etc., thereby retaining and holding the absorbent core (40) in place between the topsheet (20) and the backsheet (30).

The outward-facing surface of the backsheet that forms the underside of the absorbent article (10) may have deposit(s) of adhesive thereon. Adhesive deposit(s) or layer(s) may be provided to enable the user to adhere the article to the inside of his underpants in the lower torso and upper crotch regions thereof. A continuous layer of adhesive or a discontinuous layer of adhesive, which includes adhesive-coated region(s) and adhesive-free region(s), may be applied to the outward-facing surface of the backsheet. A total area of the adhesive-coated region(s) may be less than about 100%, preferably less than 80%, more preferably less than 60%, even more preferably less than 50% of the total surface area of the outward-facing surface of the backsheet. A total area of the adhesive-coated region(s) may be from about 30% to about 70% of the total surface area of the outward-facing surface of the backsheet. The discontinuous layer of adhesive may comprise a patterned layer of adhesive, such as an array of separate lines (separated by adhesive-free regions), spirals, or spots of adhesive. The pattern may be formed simultaneously with the applying of the adhesive layer.

A discontinuous layer of adhesive, particularly an array of separate lines of adhesive, may be preferred over a continuous layer of adhesive or a single line of adhesive (e.g., along the longitudinal axis of the article) for the conformable male incontinence absorbent articles disclosed herein. As the conformability of an absorbent article is increased and the rigidity of the absorbent article is decreased, the absorbent article may become more difficult to affix to a user's underwear, as the reduced rigidity may make it more difficult to properly position the article in the underwear and/or the article may even fold upon itself or adhere to itself, particularly if the outward-facing surface of the backsheet of the absorbent article has a continuous layer of adhesive or a total area of an adhesive-coated region(s) greater than about 75% of the total surface area of the outward-facing surface of the backsheet.

When article (10) is packaged, adhesive deposits may be covered by one or more sheets of release film or paper that covers/shields the adhesive deposits from contact with other surfaces until the user is ready to remove the release film or paper and place the absorbent article in his underpants for wear/use. The release film or paper may comprise a tab (38), as shown in FIG. 1, that extends beyond the perimeter seal (42) to enable the user to readily grip and remove the release paper. As shown in FIG. 1, the tab (38) is located on the right side of the absorbent article (10), but the tab (38) may be located anywhere along the perimeter of the article.

### Topsheet

Topsheet (20) may be formed of any suitable nonwoven web material. Referring back to the figures, the topsheet (20) is positioned adjacent a wearer-facing surface of the absorbent core (40) and may be joined thereto and to the backsheet (30) by any suitable attachment or bonding method. The topsheet (20) and the backsheet (30) may be joined directly to each other, e.g., in the perimeter seal (42) and/or may be indirectly joined by directly joining them respectively to wearer-facing and outward-facing surfaces of the absorbent core or additional optional layers included with the absorbent article.

The absorbent article (10) may have any known or otherwise effective topsheet (20), such as one which is compliant, soft feeling, and non-irritating to the wearer's skin. A suitable topsheet material will include a liquid pervious material that is comfortable when in contact with the wearer's skin and permits discharged urine to rapidly penetrate through it. A suitable topsheet may be made of any of various materials such as woven or knitted materials, nonwoven web materials, or apertured films.

The absorbent article (10) may include one or more additional layers of liquid pervious nonwoven web material between the topsheet (20) and the back sheet (30), preferably beneath the absorbent core (40), which is discussed below. The additional layer of liquid pervious nonwoven web material (25) may be useful for an absorbent article (10) that comprises a mechanically processed absorbent core (40). The additional layer of liquid pervious nonwoven web material (optionally, together with the topsheet (20)) may hold the absorbent core (40) in place during mechanical processing, which is discussed in more detail below. The additional layer of liquid pervious nonwoven web material may be directly and/or indirectly joined to the back sheet (30). The topsheet (20) and the optional one or more additional layers of liquid pervious nonwoven web material may be the same or different.

Nonlimiting examples of nonwoven web materials that may be suitable for use to form the topsheet (20), as well as additional layers of liquid pervious nonwoven that may be disposed between the topsheet (20) and the back sheet (20), include fibrous materials made from natural fibers, modified natural fibers, synthetic fibers, or combinations thereof. Some suitable examples are described in U.S. Pat. Nos. 4,950,264, 4,988,344; 4,988,345; 3,978,185; 7,785,690; 7,838,099; 5,792,404; and 5,665,452.

In some examples, the topsheet (20) and any additional layer(s) of liquid pervious nonwoven may comprise tufts as described in U.S. Pat. Nos. 8,728,049; 7,553,532; 7,172,801; 8,440,286; 7,648,752; and 7,410,683. The topsheet 20 and any additional layer(s) of liquid pervious nonwoven may have a pattern of discrete hair-like fibrils as described in U.S. Pat. No. 7,655,176 or U.S. Pat. No. 7,402,723. Additional examples of suitable materials for the topsheet and any additional layer(s) of liquid pervious nonwoven include those described in U.S. Pat. Nos. 8,614,365; 8,704,036; 6,025,535 and US 2015/041640. The topsheet and any additional layer(s) of liquid pervious nonwoven may also be formed from a three-dimensional substrate as detailed in US 2017/0258647. The topsheet, as well as any additional layer(s) of liquid pervious nonwoven, may itself comprise one or more layers, as described in US 2016/0167334; US 2016/0166443; and US 2017/0258651. The topsheet may be extensible, as defined herein.

The topsheet (20) may comprise one or more apertures to ease penetration of urine therethrough. Key properties of an apertured topshect (ATS) are open area, hole size, and hole aspect ratio. As used herein the term “hole size” refers to the average size of the open area of a single aperture, measured in units of area, for example, square millimeters. As used herein the term “open area” refers to the percentage of the total area of a web that has apertures. As used herein the term “hole aspect ratio” is the ratio of the major axis to the minor axis of a single aperture that is approximately oval shaped. The open area may be greater than about 15% and the hole size may be greater than about 2 mm². In some instances, minimum and/or maximum hole size may be important, but, unless noted otherwise herein, hole size refers to average hole size. Ideally, the holes should be circular in shape and relatively consistent in size, such that the standard deviation of the average hole size is very small. Non-round, e.g., oval-shaped, holes may also be functional, provided the hole aspect ratio, which is defined as the ratio of the major axis to the minor axis of the ellipse, is not too large. For holes having a major axis ranging from about 2 mm to about 4 mm, the hole aspect ratio is preferably less than about 6. The topsheet (20) may comprise uniformly or non-uniformly spaced apertures, each of which are approximately the same size, over the entire surface of the topsheet (20) or in one or more regions or zones of the surface of the topsheet (20). Two or more zones of apertures can be separated from one another by zones that are free of apertures. The topsheet (20) may also have two or more zones of apertures, where the two or more zones differ from one another in the size or spacing of the apertures. Examples of apertured topsheets are disclosed in U.S. Pat. No. 6,632,504.

In some examples the nonwoven web material may be a spunbond web including single-component continuous fibers, or alternatively, bi-component or multi-component fibers, or a blend of single-component fibers spun of differing polymer resins, or any combination thereof.

In order to ensure that fluid contacting the top (wearer-facing) surface of a hydrophilic topsheet will move suitably rapidly via capillary action in a z-direction to the bottom (outward-facing) surface of the topsheet where it can be drawn into the absorbent core, it may be important to ensure that the nonwoven web material forming the topsheet has an appropriate weight/volume density, reflecting suitable presence of interstitial passageways among and between the constituent fibers, through which fluid may move within the nonwoven material. A nonwoven with fibers that are consolidated too densely will have insufficient numbers and/or volume of interstitial passageways, and the nonwoven will obstruct rather than facilitate rapid z-direction fluid movement. On the other hand, a nonwoven with fibers that are not consolidated enough to provide sufficient fiber-to-fiber contact and/or sufficiently small interstitial passageways may provide insufficient potential for wicking in the z-direction via capillary action. The caliper of the topsheet material may be controlled, to balance competing needs for opacity and loft (which call for a higher caliper) vs. a limitation on the z-direction distance that discharged fluid must travel through the topsheet from the wearer-facing surface to the outward-facing surface, to reach the absorbent core below. Thus, it may be desired that the manufacture of the topsheet material be controlled to produce a topsheet material having a caliper of 0.01 mm to 0.60 mm, more preferably 0.15 mm to 0.55 mm, and even more preferably 0.30 mm to 0.45 mm.

Absorbency and wicking performance may vary according to, and may be manipulated by, the manner in which the fiber is further processed. Factors such as level of consolidation (i.e., densification) of the fiber mass in the end structure and orientations of the individual fibers within the end structure can affect absorbency and wicking performance.

Thus, for purposes contemplated herein, in combination with being imparted with a suitable basis weight, density and/or caliper as discussed above, it may be desired that nonwoven web material used to make the topsheet (20) be formed via a nonwoven web manufacturing process in which substantial numbers of the fibers are imparted with directional orientation that includes some z-direction orientation, rather than orientations predominately biased along the machine direction or x-y plane of formation of the web structure. Following any suitable processes in which fibers are distributed and laid down in a batt on a horizontal forming belt (e.g., airlaying, wetlaying, carding, etc.), additional process steps that forcibly reorient some of the fibers or portions thereof in the z-direction may be employed. Suitable process steps may include needlepunching and hydroentangling. Hydroentangling, in which an array of fine, high-velocity water jets are directed at the batt as it is conveyed past them on a foraminous belt or drum, may be desired for its effectiveness in reorienting fibers while breaking fewer fibers and creating less broken fiber lint and surface fuzz (free fiber ends extending from the main structure of the web). A vacuum water removal system (in which air is drawn through the web in a z-direction into and through a pattern of orifices or pores on a drum or belt conveying the batt, pulling the hydrojetted water with it) may be desired because it tends to create, add, open and/or clear small z-direction passageways within the fiber matrix of the web, approximately in the pattern of the orifices or pores. Without intending to be bound by theory, it is believed that an increased number of fibers (or portions thereof) oriented in the z-direction, and the z-direction passageways, increase the ability and tendency of the web to wick aqueous fluid in the z-direction. In a topsheet, this would mean that the material can more readily wick aqueous fluid from the wearer-facing surface of the topsheet to the outward-facing surface of the topsheet, i.e., directly down to the absorbent core below, and may thereby wick fluid less along x-y planar directions (causing a stain from discharged fluid to spread laterally and/or longitudinally).

### Absorbent Core

The configuration and construction of the absorbent core (40) may vary (e.g., the absorbent core (40) may have varying caliper zones, a hydrophilic gradient, a superabsorbent gradient, or lower average density and lower average basis weight acquisition zones). Further, the size and absorbent capacity of the absorbent core (40) may also be varied to accommodate a variety of wearers. However, the total absorbent capacity of the absorbent core (40) should be compatible with the design loading and the intended use of the absorbent article. The absorbent core may be web-based, meaning that the absorbent core comprises entangled masses of fibers, i.e., nonwoven fibrous webs.

The absorbent core (40) may include a plurality of layers, such as a first layer and a second layer, which may or may not be multi-functional layers. The layers may be directly joined to each other, e.g., the layers may be adhesively bonded to each other, and/or indirectly joined by directly joining them respectively to the topsheet (20) and the back sheet (40) of the absorbent article (or additional optional layers included in the article), e.g., with adhesive.

A continuous film- or coating-like deposit of adhesive may be applied to bond the topsheet and the absorbent core. However, a continuous deposit of adhesive may form a barrier that would obstruct the movement of fluid from the topsheet to the absorbent core. Accordingly, it may be preferable that, in examples in which the bonding mechanism is deposits of adhesive, the deposits are disposed in a pattern or arrangement that is discontinuous or intermittent, such that an arrangement of bonded areas interspersed with unbonded areas is created between the topsheet and the absorbent core. The adhesive may be applied to a majority of, a portion of (e.g., a bonding region), or an entirety of the wearer-facing surface area of the absorbent core (40). The adhesive may be applied in a pattern or arrangement of adhesive deposits interspersed with areas in which no adhesive is present (unbonded regions), such that the adhesive holds the two layers in close z-direction proximity, while areas remain in which no adhesive is present to obstruct z-direction fluid movement between the layers.

A dense arrangement of relatively small point bond locations may be created by spraying suitable adhesive onto one or both of the outward-facing surface of the topsheet (20) and the wearer-facing surface of the absorbent core (40) in contact with the outward-facing surface of the topsheet. If the spray nozzle is suitably configured and the rate of spray (liquid volume or weight of adhesive sprayed/time/surface area of spray coverage) is suitably regulated such that discrete spray droplets strike and adhere to the surface in discrete locations, to an extent suitably limited such that a continuous deposit or continuous film is not formed, the sprayed adhesive can form a dense random pattern of discrete point bonding locations, rather than a continuous film of adhesive that occludes pores of the underlying absorbent core or obstruct the movement of fluid from the topsheet to the underlying absorbent core.

Deposits of an adhesive/glue material, preferably an elastically extensible adhesive/glue material, may be disposed between each of the wearer-facing and outward-facing interfaces between the absorbent core (40) and the topsheet (20) and backsheet (30), respectively, or between the absorbent core (40) and other interlayered components such as, for example, the additional layer of liquid pervious nonwoven web material. The deposits may be applied via controlled spraying in the manner discussed above, so as not to create an occlusive film, but to bond the respective materials at discrete locations corresponding to deposited glue droplets, while avoiding creation of a fluid barrier, and leaving the absorbent core (40) unoccluded. Preferably, the deposits are disposed at both the upper and lower surfaces of the absorbent core (40) or a layer component thereof. Preferably, the deposits are applied over a majority of the x-y plane surface area of one or both upper and lower surfaces of the absorbent core (40). For an absorbent core that comprise multiple layers, the layers may be directly and/or indirectly joined, e.g., the layers may be bonded to teach other with adhesive, one or more of the layers may be bonded to the topsheet (20) with adhesive, and/or one or more of the layers may be bonded to an additional layer of liquid pervious nonwoven web material with adhesive.

The absorbent core (40) may comprise absorbent materials, such as super absorbent polymers (SAP). The absorbent core (40) may comprise from about 0.01% to about 20% super absorbent polymer (SAP). The use of absorbent materials in the absorbent core (40) is known to enable the construction of a thinner absorbent article having an absorption capacity like that of a thicker absorbent article. As a relatively thin absorbent core comprising absorbent materials can acquire and store large quantities of discharged body fluids, due to the ability of the absorbent materials to absorb large quantities of discharged body fluids. Alternatively, the absorbent core (40) may be substantially free of absorbent materials, such as super absorbent polymers (SAP). The inclusion of absorbent materials in the absorbent core (40) may limit the ability to mechanically process the absorbent core or the absorbent core together with one or both the topsheet 20 and backsheet 30.

The absorbent core (40) may be made from a nonwoven precursor web. The nonwoven precursor web can be formed from many processes, such as, for example, air laying processes, wetlaid processes, meltblowing processes, spunbonding processes, and carding processes. The fibers in the webs can then be bonded via spunlacing processes, hydroentangling, calendar bonding, through-air bonding and resin bonding. Some of such individual nonwoven webs may have bond sites where the fibers are bonded together. In some examples, the absorbent core (40) may comprise a web formed in a co-forming process in which plant-based fibers of finite lengths are physically blended or mixed with streams of spun fibers of longer but indefinite lengths, spun from polymeric resin, and laid down on a forming belt to form a web as described in, for example, U.S. Pat. Nos. 8,017,534; 4,100,324; US 2003/0200991; U.S. Pat. No. 5,508,102; US 2003/0211802; EP 0 333 228; WO 2009/10938; US 2017/0000695; US 2017/0002486; U.S. Pat. No. 9,944,047; 2017/0022643 and US 2018/0002848. Preferably, the absorbent core is not a fluff pulp core, which is a core comprising pulp (cellulose) fibers that have been laid down on a belt, without synthetic fibers or latex bonding or some other means to hold the fibers together under typical manufacturing line tension. Fluff pulp cores generally do not have the integrity to survive a mechanical deformation process. Also, the absorbent core is preferably free of foam material.

### Backsheet

The backsheet 30 may be positioned beneath or subjacent an outward-facing surface of the absorbent core 40 and may be joined thereto by any suitable attachment methods. For example, the backsheet 30 may be secured to the absorbent core 40 by a uniform continuous layer of adhesive, a patterned layer of adhesive, or an array of separate lines, spirals, or spots of adhesive. Alternatively, the attachment method may include heat bonds, pressure bonds, ultrasonic bonds, dynamic mechanical bonds, or any other suitable attachment mechanisms or combinations thereof. In other examples, it is contemplated that the absorbent core 40 is not joined directly to the backsheet 30.

The backsheet 30 may be impermeable or substantially impermeable by aqueous liquids (e.g., urine, menstrual fluid) and may be manufactured from a thin plastic film, although other flexible liquid impermeable materials may also be used. As used herein, the term “flexible” refers to materials which are compliant and will readily conform to the general shape and contours of the human body. The backsheet 30 may prevent, or at least substantially inhibit, fluids absorbed and contained within the absorbent core 40 from escaping and reaching articles of the wearer's clothing which may contact the absorbent article 10, such as underpants and outer clothing. However, in some instances, the backsheet 30 may be made and/or adapted to permit vapor to escape from the absorbent core 40 (i.e., the backsheet is made to be breathable), while in other instances the backsheet 30 may be made so as not to permit vapors to escape (i.e., it is made to be non-breathable). Thus, the backsheet 30 may comprise a polymeric film such as thermoplastic films of polyethylene or polypropylene. A suitable material for the backsheet 30 is a thermoplastic film having a thickness of from about 0.012 mm (0.5 mil) to about 0.051 mm (2.0 mils), for example. Any suitable backsheet known in the art may be utilized with the present invention.

Some suitable examples of materials suitable for forming a backsheet are described in U.S. Pat. Nos. 5,885,265; 4,342,314; and 4,463,045.

The backsheet 30 may have two layers: a first layer comprising a vapor permeable aperture-formed film layer and a second layer comprising a breathable microporous film layer, as described in U.S. Pat. No. 6,462,251. Other suitable examples of dual or multi-layer breathable backsheets for use herein include those described in U.S. Pat. Nos. 3,881,489, 4,341,216, 4,713,068, 4,818,600; EP 203 821, EP 710 471; EP 710 472, and EP 0 793 952.

### Caliper

The absorbent articles disclosed herein may have a caliper of about 1.5 mm to 8 mm, or 1.5 mm to 6 mm, or 2.0 mm to 5.0 mm. The caliper of the absorbent article may be uniform, e.g., across the length and width of the article, or the caliper may vary. Thicker absorbent articles (e.g., caliper greater than 8 mm) tend to be more noticeable to the wearer, as thicker articles may bunch, and tend to remind the wearer of his incontinence. Thicker absorbent articles also tend to be less discreet under clothing. However, reducing the caliper of an absorbent article may have the unintended effect of making the article more stiff and less conformable. As such, the manufacturer may wish to balance the competing objectives of providing an absorbent article that is thin and providing an absorbent article that conforms to the male anatomy without adversely affecting the fit of the underlying underwear. For purposes herein, caliper is measured using the caliper measurement method set forth below.

### Conformability

It has been learned that an absorbent article for male incontinence may be designed that more reliably and comfortably conforms to the male anatomy, in a position to better intercept and capture urine flow in the absorbent material before it can escape and permit expansion of the absorbent components, initially and over a reasonable duration of wear/use of the pant. It has also been learned that an absorbent article may be designed with a select conformability to comfortably hold the absorbent article against/in contact with the topography of the male anatomy.

It has also been discovered that the in wear comfort of such an absorbent article affixed to a wearer's underwear is enhanced when the absorbent article is conformable and moves with the underwear. In contrast, it is believed that when a stiff or inflexible absorbent article is affixed to a wearer's underwear, the absorbent article and the underlying underwear form a laminate that is more stiff and less comfortable than the underwear alone. Thus, an absorbent article that conforms to the male anatomy and moves with the wearer's underwear may be preferred. However, the conformability of the article is also preferably selected so that the article can be easily affixed to the underwear; if the conformability of the article is too low, then the article may be floppy and, therefore, difficult to affix to the underwear.

The absorbent articles disclosed herein may exhibit a Conformability Force at 5 mm less than about 3500 N/m, preferably less than about 2,200 N/m, more preferably less than 1800 N/m, even more preferably less than about 1,000 N/m or 900 N/m, and greater than about 260 N/m, preferably greater than about 300 N/m, more preferably greater than about 400 N/m, and an average dry caliper of about 2 mm to about 7 mm, preferably about 2 mm to about 5 mm, more preferably about 2 mm to about 4 mm.

### Deformation

It has been discovered that deformation of at least the absorbent core 40, or the absorbent core 40 together with the topsheet 20, or the absorbent core together with a layer of liquid pervious nonwoven carrier web (as a support layer), or the absorbent core 40 together with the topsheet 20 and a layer of liquid pervious nonwoven carrier web (which, together with the topsheet 20, may hold the absorbent core 40 in place during deformation), in the manner described herein, can provide a number of unexpected benefits. The backsheet may also be mechanically processed, as long as the mechanical processing does not impact the integrity and liquid impermeability of the backsheet. It may be advantageous not to form deformations in the liquid impermeable backsheet to maintain the integrity of the backsheet.

A deformation process known as “incremental stretching” involves passing a web through a nip between a pair of rollers, the rollers having mating features that cause incremental sections of the web to be stretched across lines coincident with features of the rollers. The different processes that utilize intermeshing rolls to incrementally stretch a web producing protrusions and undeformed regions include ring rolling, SELF, and Nested SELF. Each of the processes comprise a roll that has male elements that extend outward from the surface of the roll, and cavities/recesses designed to receive the male elements. Incremental stretching processes may be applied to not only a single web layer, but a composite web of a plurality of layers including components of an absorbent article, to beneficial effect.

Non-limiting examples of incremental stretching processes and equipment are disclosed in U.S. Pat. No. 6,383,431. Ring Rolling is described in U.S. Pat. No. 5,626,571 and can produce protrusions that resemble corrugations and extend the full length of the product or the full length of the region that is deformed.

The terms “SELF” and “SELF”ing,” refer to Procter & Gamble technology in which SELF stands for Structural Elastic Like Film. While the process was originally developed for deforming polymer film to have beneficial structural characteristics, it has been found that the SELF'ing process can be used to produce beneficial structures in other materials such as nonwovens. Processes, apparatuses, and patterns produced via SELF are illustrated and described in U.S. Pat. Nos. 5,518,801; 5,691,035; 5,723,087; 5,891,544; 5,916,663; 6,027,483; and 7,527,615 B2. One such SELF process is described in (Tufted Fibrous Web—U.S. Pat. No. 7,829,173). The term “tuft,” as used herein, refers to a protrusion in the surface of a nonwoven web comprising a plurality of looped fibers extending from the surface of the web. Tufts may have a tunnel-like configuration which may be open at both of their ends. In materials such as airlaid nonwovens (which may be used for cores), SELF′ing can produce elongated tent-like structures, which may or may not be open at their ends.

Another SELF process is nested SELF. Nested SELF is a process described in U.S. Pat. Nos. 10,045,888 and 10,500,826, which produces three-dimensional protrusions having a dome-like shape on one side of the material and apertures or base openings formed in the opposing side of the material. The domes can have different shapes, such as circular-shaped domes, oval-shaped domes, and diamond-shaped domes. Nested SELF is described in more detail in U.S. Pat. No. 11,020,938.

Plastic deformation from the incremental stretching process can result in different web properties in more deformed regions when compared to regions with less or even no deformation. Fibers can be pulled apart or separated in the more deformed region, resulting in lower density, lower basis weight, lower caliper, or lower integrity versus the adjacent less deformed region. For webs with extensible fibers, more deformed regions might contain fibers that are thinner or more oriented in the direction of stretching.

The plastic deformation of a web depends on the process, the required apparatus, and also on the properties of the web, i.e. apparent elongation of the fibers, fiber mobility, ability to deform and stretch in the area where the deformations are formed, ability to undergo plastic deformation which sets after exiting a first and a second roller, or springing partially back, due to elastic recovery. The deformation process may comprise engaging the web between a first and second roller, such that a plurality of deformations are obtained.

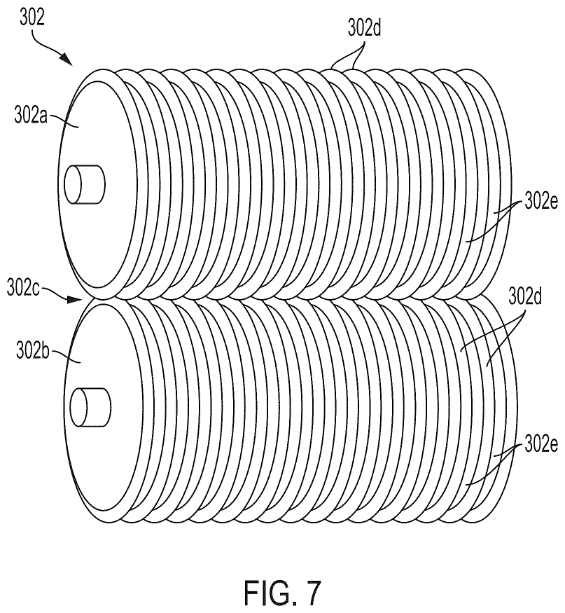

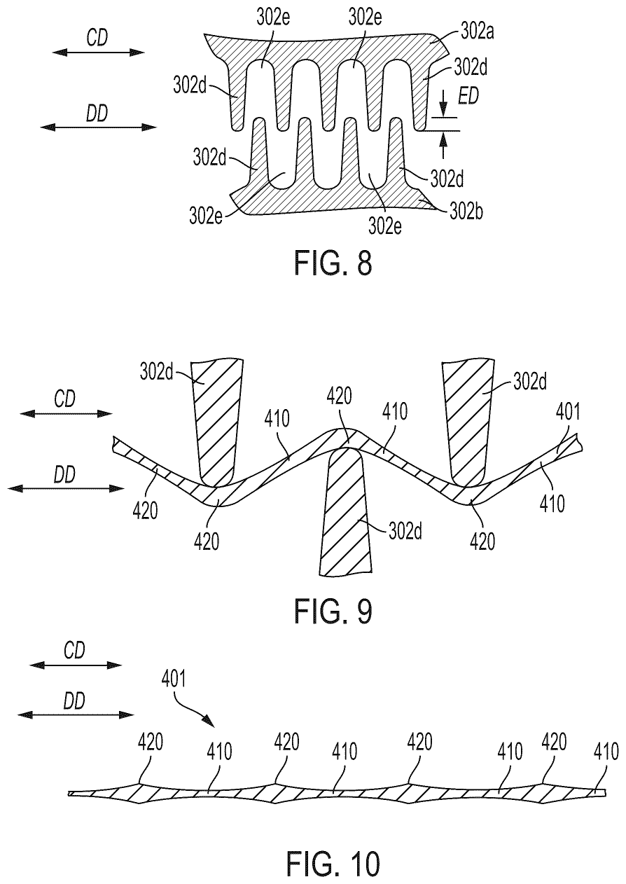

The roller configuration shown in FIG. 7 is sometimes known as a “ring rolling” configuration, or “ring rollers.” Referring to FIGS. 7-10, mating rollers (302a, 302b) may be provided with respective circumferential ridges 302d separated by circumferential grooves (302c). The rollers may be configured such that the ridges of one engage with the grooves of the other to a desired engagement depth ED (FIG. 8). Upon passage of a precursor web (400) being conveyed along a machine direction MD through a nip (302c) between the engaged rollers, it will be stretched or strained along a cross direction CD between the respective ridges (302d) of each roller. If the engagement depth ED is sufficient, one or more layer components of the precursor web (400) will stretch beyond their yield points to plastic deformation, along machine direction lines, as the web (400) passes through the nip, resulting in a deformed web (401). FIGS. 9 and 10 show the resulting regions with more deformation (410) with a lower basis weight alternating with regions of less or no deformation (420). In this configuration, the direction of deformation DD is aligned or approximately parallel with the cross direction CD.

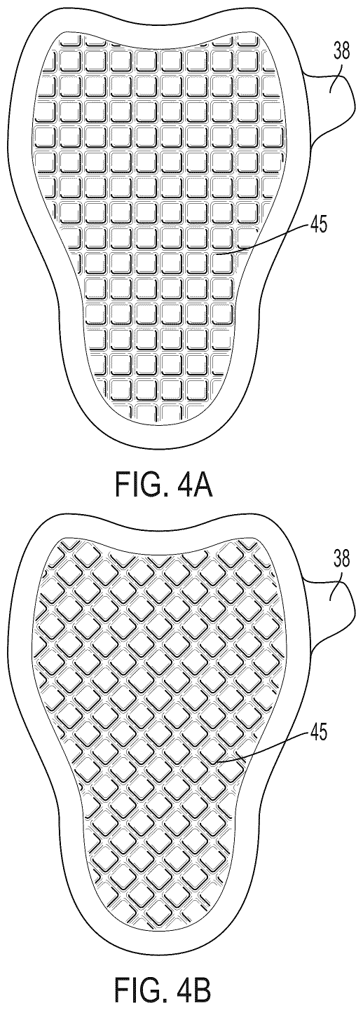

After undergoing a process of deformation, using the roller configuration shown in FIG. 7, the deformations may resemble spaced apart regions of reduced basis weight or reduced fiber integrity that run substantially continuously and parallel in at least 1 direction, but often in 2 directions, as exemplified in the absorbent articles in FIGS. 4A-4B.

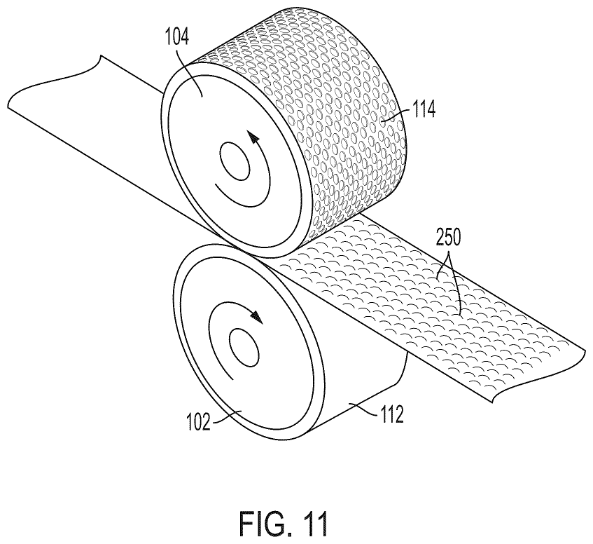

In the roller configuration shown in FIG. 11, the first and second rollers (102, 104) are non-deformable, meshing, counter-rotating rolls that form a nip therebetween. The precursor web(s) is/are fed into the nip (106) between the rollers (102, 104). Although the space between the rollers (102, 104) is described herein as a nip, in some cases, it may be desirable to avoid compressing the precursor web(s) to the extent possible. The first roller (“male roller”) (102) has a surface comprising a plurality of discrete, spaced apart male forming elements or male elements (112). The male forming elements are spaced apart in the machine direction and in the cross-machine direction. The term “discrete” does not include continuous or non-discrete forming elements such as the ridges and grooves on ring rolls (or “corrugated rolls”) which have ridges that may be spaced apart in one, but not both, of the machine direction and in the cross-machine direction. The male forming elements (112) have a base that is joined to and/or integral with the first roller (102), a top that is spaced away from the base, and sides that extend between the base and the top of the male forming elements (112). The male forming elements (112) may also have a transition portion or region between the top and the sides. The male elements (112) may also have a plan view periphery, and a height, which is measured from the base to the top.

The tops of the discrete elements on the male roller may have relatively large surface areas (e.g., from about 1 mm to about 10 mm in width, and from about 1 mm to about 20 mm in length) for creating a wide deformation. The male elements (112) may, thus, have a plan view aspect ratio (ratio of length to width) that ranges from about 1:1 to about 10:1. For the purpose of determining the aspect ratio, the larger dimension of the male elements (112) will be considered the length, and the dimension perpendicular thereto will be considered the width of the male element. The male elements 112 may have any suitable configuration. The base and the top of the male elements (112) may have any suitable plan view configuration, including but not limited to: a rounded diamond configuration, as shown in FIG. 11, an American football-like shape, triangle, circle, clover, a heart-shape, teardrop, oval, or an elliptical shape. The configuration of the base and the configuration of the top of the male elements (112) may be in any of the following relationships to each other: the same, similar, or different. The top of the male elements (112) can be flat, rounded, or any configuration therebetween. The top on the male forming elements (112) may have a rounded diamond shape, with vertical sidewalls and a radiused or rounded edge at the transition between the top and the sidewalls of the male forming element (112).

The region between the top and the side walls of the male forming elements (112) may also be of any suitable configuration. This region between the top and the side walls of the male forming elements (112) can be in the form of a sharp edge in which case there is zero, or a minimal radius where the side walls and the top of the male forming elements meet. That is, the region between the top and the side walls of the male forming elements (112) may be substantially angular, sharp, non-radiused, or non-rounded. In other embodiments, the region between the top and the side walls of the male forming elements (112) can be radiused, or alternatively beveled. Suitable radiuses include, but are not limited to: zero (that is, the transition forms a sharp edge), 0.01 inch (about 0.25 mm), 0.02 inch (about 0.5 mm), 0.03 inch (about 0.76 mm), 0.04 inch (about 1 mm) (or any 0.01 inch increment above 0.01 inch), up to a fully rounded male forming elements (112).

A second roller (“female roller”) (104) may have a surface comprising a plurality of cavities or recesses (114) in the second roller (104). The recesses (114) may be aligned and configured to receive the respective male forming elements (112) therein. Hence, each recess (114) of the second roller (104) may be sufficiently large to be able to receive each respective male forming element (112) of the first roller (102). The recesses (114) may have a similar shape as the male forming elements (112). The depth of the recesses (114) may be greater than the height of the male forming elements (112).

The first and second rollers (102, 104) may be further defined by a depth of engagement (DOE) which is a measure of the level of intermeshing of the first and second rollers (102, 104). The depth of engagement (DOE) may be measured from the top of the male forming element (112) to the outermost portion of the surface of the second roller (104) that is not within a recess (114). The depth of engagement (DOE) may range from 1.5 mm to 5.0 mm or from 2.5 mm to 5.0 mm or from 3.0 mm to 4.0 mm. The first and second rollers (102, 104) may be defined by a clearance between the first and second rollers (102, 104). The clearance is the distance between the side wall of the male forming element (112) and the side wall of the recess (114). The clearance may range from 0.1 mm to 2 mm or from 0.1 mm to 1.5 mm from 0.1 mm to 1 mm. The deforming rollers may have engaging teeth having an MD pitch of 2.3 teeth per cm, a CD pitch of 2.6 teeth per cm, and an engagement depth of 0.2 cm.

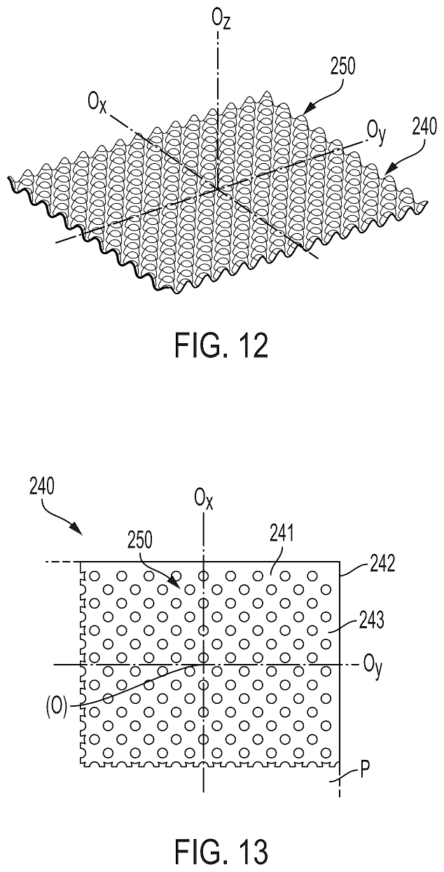

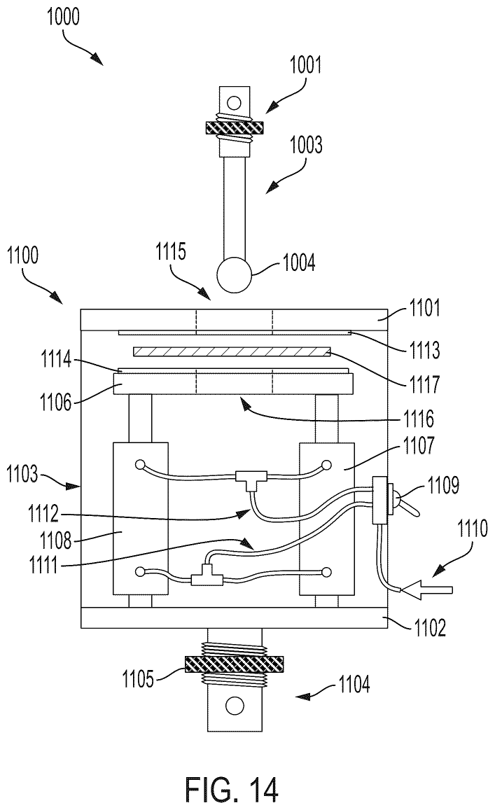

After undergoing a process of deformation, using the roller configuration shown in FIG. 11, the deformed web may comprise land areas 243 and deformations comprising three-dimensional protrusions 250, as shown in FIGS. 12-13. The land areas 243 may be substantially flat areas. The deformed web may comprise a majority of three-dimensional protrusions 250 having a first Z-directional height (O, in FIG. 12). The majority of the three-dimensional protrusions 250 protrudes from the land areas 243 of the three-dimensional absorbent core forming a base and an opposed distal portion from the land areas. The opposed distal portion of the majority of three-dimensional protrusions 250 extends to a distal end which forms a top peak which is spaced away from the base of the majority of three-dimensional protrusions 250. The base of the majority of three-dimensional protrusions can be defined as the perimeter, which for circular protrusions, is the circumference, where each protrusion of the majority of three-dimensional protrusions 250 starts to protrude outwardly from the land areas of the deformed web.

Referring to FIG. 13, the deformed web forms a first surface 241 and a second surface 242. The majority of three-dimensional protrusions 250 extend outward from the second surface 242 of the deformed web. The majority of three-dimensional protrusions 250 are surrounded by a plurality of land areas 243 of the deformed web. The plurality of land areas 243 of the deformed web may be located on the first surface 241 of the deformed web. The majority of three-dimensional protrusions 250 of the deformed web may form a three-dimensional surface on the second surface 242 of the deformed web.

The majority of three-dimensional protrusions 250 can be hollow. When viewing from the first surface 241 of the deformed web, the majority of three-dimensional protrusions 250 may protrude from the land areas 243 of the deformed web in the same direction. Alternatively, the three-dimensional protrusions 250 may protrude from the land area 243 of the deformed web in opposite directions. The plurality of land areas 243 and the plurality of three-dimensional protrusions 250 together form a three-dimensional deformed web. The majority of the three-dimensional protrusions 250 may be generally dome-shaped. Two or more adjacent three-dimensional protrusions 250 may be separated from each other by one or more land areas 243 in a direction generally perpendicular to the longitudinal axis or in a direction generally parallel to the longitudinal axis of the three-dimensional deformed web. The majority of the three-dimensional protrusions 250 extending outwardly from the first surface 241 of the three-dimensional deformed web may represent at least about 20%, or at least about 30%, or at least about 40%, or more than about 50%, or more than about 70%, or more than about 80%, but not more than about 95% of the total area of the three-dimensional deformed web.

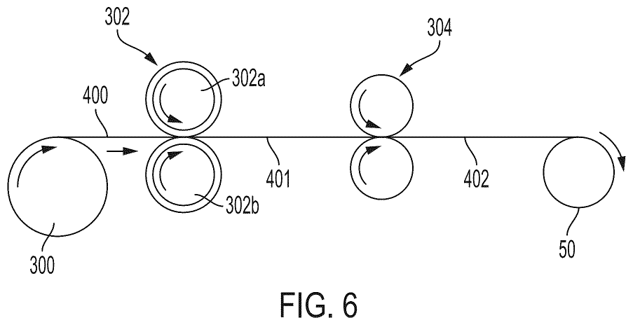

Referring now to FIG. 6, a precursor web (400) (provided on supply roll (300)) comprising an absorbent core may be sequentially passed through the nips between a pair of deforming rollers (302), causing the web to be incrementally stretched, as described above, along one direction (in this particular non-limiting example, the cross direction). The deformed web (401) may then, optionally, be passed through a pair of consolidating rollers (304), as shown in FIG. 6, to form a consolidated web (402), which is then wound up on take-up roll (50).

It has been discovered that, following such deformation processes, the deformed absorbent article is dramatically more flexible and pliable, making it more capable of bending around and conforming to a non-ruled surface. Without intended to be bound by theory, it is believed that an absorbent article having one or more layer components so processed will be dramatically more comfortable to a wearer/user of the product, and more effective at intercepting discharges of body fluids, as a result of greater conformability to body features.

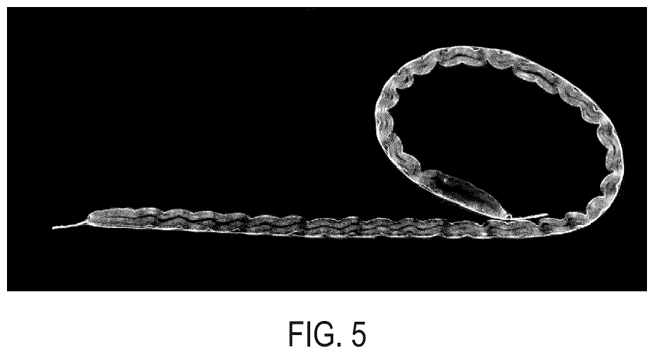

It is believed that the enhanced conformability is attributable to the creation of regions of, for example, reduced caliper, reduced fiber density, reduced basis weight, intermittent voids, or reduced fiber integrity within the absorbent core, along the lines of deformation, as suggested by FIG. 5. Further, when the precursor web 400 that is passed between the deforming rollers includes not only an absorbent core but also a topsheet, a nonwoven carrier web, and/or a backsheet, all of the layers may be deformed to varying extents depending upon their deformability, and all of the layers together as a composite can thereby be imparted with expanded size in the x-y directions, increased conformability, flexibility and pliability. Importantly, the deformed composite web has enhanced capability to bend around and more closely conform to non-ruled, curved surfaces such as the surfaces of human body features. The extent of this conformability is reflected in measurements that may be taken using the Conformability Force Measurement Method described below.

The materials of the respective layers of the absorbent article, such as the topsheet 20, the optional additional layer of liquid pervious nonwoven carrier web, the absorbent core 40, and the backsheet 30 (if included) may be selected for having properties, and the engaging features and engagement depths of the deforming rollers may be configured and adjusted such that, for example, the topsheet and/or backsheet only stretch elastically but not plastically, or alternatively plastically but not to breakage, as they pass through the respective nips between the deforming rollers, while the material(s) of the absorbent core can simultaneously be stretched plastically or even to breakage/fracture, in an orderly manner and pattern reflecting the patterns of ridges and grooves on the deforming rollers. In such a configuration, the combination of materials to form the absorbent article, as a composite, may be imparted with conformability and enhanced pliability. A non-limiting example of an absorbent article in the form of a male incontinence absorbent article having undergone such deformation is schematically depicted in FIG. 1.

FIG. 2 is a schematic lateral cross section of the article shown in FIG. 1, taken through line 2-2 in FIG. 1, showing how the layers of the article are stacked. FIG. 2 schematically depicts an example of potential effects resulting from the deformation of the absorbent core 40 of the article, where the topsheet 20 and the backsheet 30 do not undergo deformation. Though not shown in FIG. 2, the absorbent core 40 may comprise multiple layers, some or all of which undergo deformation. The absorbent core may also be combined with a nonwoven carrier web to form a laminate, where the laminate undergoes deformation and the deformed laminate is then assembled into the final article. For an absorbent core comprising more than one layer, such as a first layer comprising fibrous nonwoven web and a second layer comprising fibrous nonwoven web, the fibers of the first layer of nonwoven web may be driven into or driven through a plane occupied by the second layer of nonwoven web.

Without being bound by theory, it is believed that the deformations, e.g., three-dimensional protrusions, in the absorbent core increase the conformability of the absorbent article by decreasing the bending stiffness of the article, which may ultimately enhance the in-wear comfort of the article. The decrease in bending stiffness may be due to a reduction in caliper, a reduction in basis weight, a reduction in fiber integrity, a void space created by a three-dimensional protrusion or another physical change, depending on the process used to deform the absorbent core. For example, embossing may result in reduced caliper, which may allow the embossed material to bend more easily at the regions of reduced caliper. Similarly, intermeshing processes that strain the web and result in regions of reduced basis weight, reduced fiber density, or reduced fiber integrity may allow the processed material to bend more easily in these regions. Still other processes, such as the process commonly referred to as the “SELF” process (where SELF stands for Structural Elastic Like Film), as described in U.S. Pat. No. 8,021,591, creates elongated tent-like three-dimensional protrusions resulting in voids or spaces between deformations that allow the processed material to bend and buckle easily. As shown in FIG. 5, when a processed absorbent article that includes deformations is bent, the deformations are compressed, while the topsheet and backsheet conform to the absorbent core structure with little or no buckling or pulling away from the absorbent core structure. FIG. 5 shows regularly spaced deformed regions with lower caliper in the processed absorbent article that allow the article to easily bend or buckle. This phenomenon is also seen in processed absorbent articles with deformed regions containing a reduction in basis weight, reduction in fiber integrity or voids created by three-dimensional protrusions.

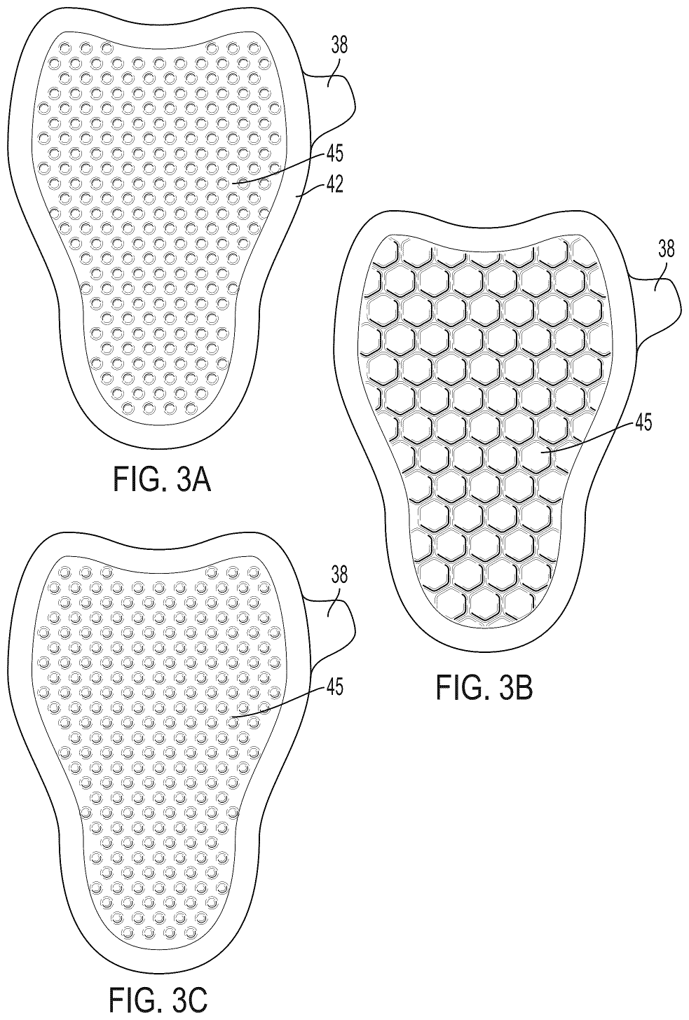

As shown in FIGS. 3A-3C and 4A-4B, the deformations may be formed in different shapes, sizes, and orientations. Also, the majority of the wearer-facing surface area of the absorbent article 10 may include deformations 45, as shown in FIG. 3A, where the perimeter seal 42 does not include deformations. Alternatively, the entirety of the wearer-facing surface area of the absorbent article or a portion(s) or zone(s) of the wearer-facing surface area of the absorbent article (10) may include deformations 45. A zone of deformations having an average radius or other largest dimension of 1.0 mm to 4.0 mm, or 1.5 mm to 3.5 mm, may be included, within, for example, the area occupied by the rearward end region 65 or the forward end region 60. The deformations may be present in the majority of, portion of, or entirety of the wearer-facing surface area at a numerical density of 3.0 to 9.0 deformations per cm², or 4.0 to 8.0 deformations per cm², with a CD spacing of 2-15 mm, preferably 3-10 mm, and an MD spacing of 2-15 mm, preferably 3-10 mm. The recesses in the mating female roll also had a rounded diamond shape, similar to that of the male roll, with a clearance between the rolls of 0.53-1.09 mm (0.021-0.043 inch). The average size, shape, numerical density, spacing, and surface area occupied by the deformations may be selected by the manufacturer to optimize the conformability of the absorbent article 10.

To study the effects of the deformation processes described above, example and comparative products are constructed and tested (the construction of each such product is detailed in Table 1). The resulting products, along with examples of various currently marketed products designed for male incontinence, are measured for conformability using the Conformability Force Measurement Method. Examples 1-6 are products according to the present disclosure, while Examples 7-16 are comparative examples.

Examples 1-5 and 8 are provided to a group of 11-12 panelists for a usage test. The panelists rate Examples 1-5 as providing outstanding performance, while being unnoticeable (to the panelist) during wearing. Comparative example 8 is rated by the panelists as being unusable: the panelists report that the products are too thin or flimsy, which makes inserting and affixing the products to their underwear difficult; the lack of rigidity of the products makes it difficult to properly position the products in the underwear (before the adhesive layers of the products contact the underwear and affix the products). The panelists also report that the products of comparative example 8 fold upon themselves and adhere to themselves, which renders the products unusable.

TABLE 1
Examples

| | | Conform- | Dry | Wet | Dry | Wet | Swelling | Max. |
|---|---|---|---|---|---|---|---|---|
| | | ability | Caliper | Caliper | Wt. | Wt. | Ratio | Capacity |
| | Product Description | N/m | mm | mm | g | g | — | g |
| 1 | Absorbent Core: 25 gsm | 820 | 2.3 | — | — | — | — | — |
| | spunbond¹ + 100 gsm | | | | | | | |
| | PP/pulp core + 25 gsm | | | | | | | |
| | spunbond¹, where all 3 | | | | | | | |
| | layers are deformed using a | | | | | | | |
| | discrete deformation | | | | | | | |
| | pattern, like the pattern | | | | | | | |
| | shown in FIG. 3A, with a | | | | | | | |
| | repeat length of | | | | | | | |
| | approximately 14 mm. A 23 | | | | | | | |
| | gsm impermeable | | | | | | | |
| | backsheet² is attached to the | | | | | | | |
| | side with protrusions. | | | | | | | |
| 2 | Absorbent Core: 25 gsm | 586 | 1.8 | — | — | — | — | — |
| | spunbond + 100 gsm | | | | | | | |
| | PP/pulp core + 25 gsm | | | | | | | |
| | spunbond, where all 3 layers | | | | | | | |
| | are deformed using a | | | | | | | |
| | discrete deformation | | | | | | | |
| | pattern, like the pattern | | | | | | | |
| | shown in FIG. 3B, with a | | | | | | | |
| | repeat length of app 34 mm. | | | | | | | |
| | A 23 gsm impermeable | | | | | | | |
| | backsheet is attached to the | | | | | | | |
| | side opposite the side with | | | | | | | |
| | protrusions. | | | | | | | |
| 3 | Absorbent Core: 25 gsm | 832 | 2.5 | — | — | — | — | — |
| | spunbond + 100 gsm | | | | | | | |
| | PP/pulp core + 25 gsm | | | | | | | |
| | spunbond, where all 3 layers | | | | | | | |
| | are deformed using a | | | | | | | |
| | discrete deformation | | | | | | | |
| | pattern, like the pattern | | | | | | | |
| | shown in FIG. 3C, with a | | | | | | | |
| | repeat length of app 14 mm. | | | | | | | |
| | A 23 gsm impermeable | | | | | | | |
| | backsheet is attached to the | | | | | | | |
| | side opposite the side with | | | | | | | |
| | protrusions. | | | | | | | |
| 4 | Absorbent Core: 25 gsm | 871 | 3.3 | — | — | — | — | — |
| | spunbond + 160 gsm | | | | | | | |
| | BICO/pulp blend³ + 25 gsm | | | | | | | |
| | spunbond, where all 3 layers | | | | | | | |
| | are deformed using a | | | | | | | |
| | continuous deformation | | | | | | | |
| | pattern with a repeat length | | | | | | | |
| | of app 5 mm, like the | | | | | | | |
| | pattern shown in FIG. 4A. | | | | | | | |
| | A 23 gsm impermeable | | | | | | | |
| | backsheet is attached to | | | | | | | |
| | either side. | | | | | | | |
| 5 | Absorbent Core: 25 gsm | 712 | 2.7 | — | — | — | — | — |
| | spunbond + 160 gsm | | | | | | | |
| | BICO/pulp blend + 25 gsm | | | | | | | |
| | spunbond, where all 3 layers | | | | | | | |
| | are deformed using a | | | | | | | |
| | continuous deformation | | | | | | | |
| | pattern with a repeat length | | | | | | | |
| | of app 5 mm, like the | | | | | | | |
| | pattern shown in FIG. 4B | | | | | | | |
| | (where the deformed | | | | | | | |
| | continuous areas are each at | | | | | | | |
| | an angle of approximately | | | | | | | |
| | 45 degrees relative to the | | | | | | | |
| | longitudinal axis of the | | | | | | | |
| | product). A 23 gsm | | | | | | | |
| | impermeable backsheet is | | | | | | | |
| | attached to either side. | | | | | | | |
| 6 | Absorbent Core: 25 gsm | 870 | 2.5 | 2.6 | 8.9 | 80 | 1.0 | 71 |
| | spunbond + 160 gsm | | | | | | | |
| | BICO/pulp blend + 25 gsm | | | | | | | |
| | spunbond, where only the | | | | | | | |
| | 160 gsm BICO/pulp blend | | | | | | | |
| | layer is deformed. | | | | | | | |
| 7 | Absorbent Core: 25 gsm | 4848 | 2.6 | 2.6 | 3.3 | 39.6 | 1.0 | 36.3 |
| | spunbond + 160 gsm | | | | | | | |
| | BICO/pulp blend + 25 gsm | | | | | | | |
| | spunbond, where neither the | | | | | | | |
| | absorbent core nor any of its | | | | | | | |
| | constituent layers are | | | | | | | |
| | deformed. | | | | | | | |
| 8 | Absorbent Core: 25 gsm | 250 | 1.81 | — | — | — | — | — |
| | spunbond + 100 gsm | | | | | | | |
| | PP/pulp core + 25 gsm | | | | | | | |
| | spunbond, where all 3 layers | | | | | | | |
| | are deformed using a | | | | | | | |
| | continuous deformation | | | | | | | |
| | pattern with a repeat length | | | | | | | |
| | of app 5 mm, like the | | | | | | | |
| | pattern shown in FIG. 4A. | | | | | | | |
| | A 23 gsm impermeable | | | | | | | |
| | backsheet is attached to the | | | | | | | |
| | side opposite the side with | | | | | | | |
| | protrusions. | | | | | | | |
| 9 | Certainty Shield | 2,380 | 3.9 | 9.0 | 6.2 | 101 | 2.3 | 95 |
| 10 | Depends Guard | 2,390 | 8.1 | 15.0 | 25 | 370 | 1.8 | 345 |
| 11 | Depends Shield | 4,740 | 2.7 | 7.9 | 4.9 | 65 | 2.9 | 60 |
| 12 | Tena Black Shield | 3491 | 4.4 | 11.2 | 6.5 | 117.7 | 2.6 | 111.1 |
| 13 | Tena Intimates Moderate | 2,640 | 9.1 | 19.4 | 14 | 239 | 2.1 | 225 |
| 14 | Tena Level 1 | 1,160 | | | | | | |
| 15 | Equate Assurance | 2,380 | 9.4 | 19.7 | 20 | 319 | 2.1 | 300 |
| 16 | Up & Up Mens guards | 2,390 | 7.8 | 16.6 | 25 | 379 | 2.1 | 354 |

¹A spunbond polypropylene nonwoven material having a basis weight of 25 gsm.
²A backsheet formed of a polyethylene film having a basis weight of 23 gsm.
³An airlaid cellulosic (wood pulp) fiber blended with staple bicomponent fibers of a polyethylene/polypropylene sheath core configuration, having a basis weight of 160 gsm.

The data appearing in Tables 1 and 2 above reflect that the inventive prototypes exhibited dramatically greater conformability, using the Conformabililty Force Measurement Method, than any of the current market products measured. The data were consistent with tactile and visual impressions from viewing and handling the products. The inventive absorbent articles in Table 2 also had some of the lowest dry caliper measurements of all the products tested, while providing greater conformability.

### Test Methods

### Conformability Force Measurement Method

Measurements made using this Conformability Force Measurement Method reflect the extent to which a composite web material (i.e., combination, assembly or laminate of web materials) will resist bending and stretching around a non-ruled surface. In this method, the non-ruled surface is a spherical ball 1004 that is 25.4 mm in diameter.

The Conformability Force Measurement Method is performed on a constant rate of extension tensile tester (a suitable instrument is MTS Insight tensile tester operated under TestSuite software, MTS Systems Corp, Eden Prairie, MN, or equivalent) using custom fixtures and an appropriate capacity load cell. All testing is performed in a laboratory controlled at 23° C.±2° C. and 50%±2% relative humidity.

Referring to FIG. 13, the bottom fixture 1100 is a pneumatic clamping system used to secure the specimen 1117 horizontally for testing. The fixture is built of a box 1103 made of 6.4 mm Plexiglass with a top 1101 and a bottom 1102 both made of 9.5 mm thick aluminum plates. The bottom 1102 plate is attached to the bottom mount of the tensile tester via an adapter 1104 and locking collar 1105 used to secure the fixture orthogonal to the mount of the tensile tester. Interior to the box a movable plate 1106 made of 9.5 mm thick aluminum is attached to two pneumatic cylinders 1107 and 1108 used to raise and lower the test specimen 1117. A rubber gasket 1113 is affixed on the bottom side of the top plate 1101 with a matching rubber gasket 1114 affixed to the top of the movable plate 1106. A circular 44.45 mm diameter, vertically oriented orifice 1115 passes through the longitudinal and lateral center of the top plate 1101 and gasket 1113. A corresponding circular 44.45 mm diameter orifice 1116 is vertically aligned with orifice 1115 and passes through the movable plate 1106 and gasket 1114. Pressurized air 1110 is provided to a switch 1109 which is fluidly connected via tubing 1111, 1112 to the cylinders 1107, 1108 and is used to raise and lower the movable plate 1106. The air pressure is sufficient to hold the sample securely without slippage for testing.

The upper fixture includes a cylindrical plunger 1003 terminating in the spherical ball 1004 that has a diameter of 25.4 mm. The plunger has an adapter 1001 compatible with the mount on the load cell capable of securing the plunger orthogonal to the top plate 1101 of the bottom fixture. When the fixtures are assembled with the tester, the ball 1004 is vertically centered over orifices 1115 and 1116, and the center of the ball travels along a vertical path coincident with the vertical axes of the orifices. The gage length is set at 10 mm between the bottom surface of the ball 1004 and the bottom surface of gasket 1113.

The instrument is programmed for compression mode. The crosshead is lowered at a rate of 500 mm/min for 15 mm and then returned to its original position. Data from the force and distance channels is recorded at a rate of 100 Hz during the downward stroke.

Test sample products are conditioned in a laboratory controlled at 23° C.±2° C. and 50%±2% relative humidity. Each sample is placed on a flat bench, with the topsheet facing upward. The longitudinal axis of the sample is identified. From the top edge of the absorbent core, along the longitudinal axis, 45 mm is measured, and the location is marked, e.g., with a dot. This location is the center of the measurement site. If present, a release paper (or an adhesive coversheet) is removed from the sample. The marked center of the measurement site is centered within the opening 1116 in the moveable plate 1106 and gasket 1114. Prior to securing the sample between the gaskets 1113, 1114, the sample is gently pulled taut with approximately equal tension applied along two perpendicular directions, only to an extent sufficient eliminate sagging over the opening 1116. After pulling the sample taut, the switch 1109 is activated to admit air into the cylinders 1107, 1108 and thereby raise the movable plate 1106, to secure the sample between the gaskets 1113, 1114. The force and crosshead channels are zeroed, and the program is started. After the test, the movable plate is lowered and the sample is removed prior to analyzing the next sample. Testing is performed on seven (7) replicate samples.

A graph of force (N) verses displacement (mm) is constructed for each test run. The peak force (N) from the graph is recorded to the nearest 0.01 N. The Conformability Force is calculated as the greatest slope of the curve utilizing a line segment that is at least 20% of the maximum force and recorded to the nearest 0.1 N/m. The Conformability Force is recorded as the average obtained from seven replicate specimens to the nearest 1 N/m.

### Computerized Tomography (CT) Scan Method

Computerized Tomography (CT) data are obtained with a FlashCT (Hytec, Los Alamos, NM) μCT system containing a Perkin Elmer XRD1621 flat panel detector. The acquisition parameters are 150 kVp, 250 μA, 250 mS integration time, and 8× frame averaging. The reconstructed volume has an isotropic resolution of approximately 100 μm and a field of view of approximately 160×180×60 mm. Visualization is done using Avizo software by Thermo Fisher Scientific.

### Caliper Measurement Method

Dry and wet caliper of the absorbent articles is measured using a ProGage Thickness Tester (Thwing-Albert Instrument Company, West Berlin, N.J.) with a foot diameter of 56.4 mm (area of 25 cm²) at a pressure of 0.5 kPa or equivalent. The dry and wet mass of each article is measured using an analytical balance accurate to 0.1 g. All testing is performed in a laboratory controlled at 23° C.±2° C. and 50%±2% relative humidity.

A 0.9% saline (w/w) solution is prepared and approximately two gallons of the saline solution is added to a 5 gallon bucket, where each article is immersed in the saline solution for the wet portion of the caliper test. The solution can be used to wet up to 20 articles, but then is replaced with a fresh 0.9% saline solution.

All primary and secondary packaging of the articles is removed. If present, any release paper is removed from the backsheet of the article. Test articles are conditioned in a laboratory controlled at 23° C.±2° C. and 50%±2% relative humidity for 1 hour before testing.

The article (with the release paper removed) is then flattened against a benchtop with the top sheet facing upward. The boundary of the absorbent core, around the entire perimeter of the article, is determined and the longitudinal and lateral center point of the absorbent core is marked.

The caliper is zeroed and the article is placed on the anvil of the caliper, top sheet surface facing upward, with the marked center point centered under the pressure foot. The foot is lowered at 0.076 cm/sec to an applied pressure of 0.5 kPa. A reading is taken after a 3-second dwell time and the foot is raised. The dry caliper is recorded to the nearest 0.01 mm. The analytical balance is zeroed, the article is placed on the balance, and the dry mass of the article is recorded to the nearest 0.1 g.

For wet caliper measurement, each article is immersed in the 0.9% saline solution for five minutes±10 s, while gently agitating the article intermittently. The article is then removed from the solution and suspended vertically from its top edge for 30 s±2 s. The caliper is zeroed and the article is immediately placed on the anvil of the caliper, top sheet surface facing upward, with the marked center point centered under the pressure foot. A reading is taken after a 3-second dwell time and the foot is raised. The wet caliper is recorded to the nearest 0.01 mm. The analytical balance is zeroed, the article is placed on the balance, and the wet mass of the article is recorded to the nearest 0.1 g. The capacity of the article is calculated by subtracting the dry mass from the wet mass and is reported to the nearest 0.1 g. The wet article is then discarded and the surfaces of the caliper and the pan of the balance are wiped.

This procedure is repeated in like fashion for five replicate articles and the dry caliper, wet caliper, and capacity are reported as the algebraic average of the five replicates.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent pad configured for placement within a wearer's underpants adjacent the wearer's body, comprising:
a) a liquid permeable topsheet;
b) a liquid impermeable backsheet; and
c) an absorbent core disposed between the topsheet and the backsheet, the absorbent core comprising a fibrous nonwoven web, a batt or web of cellulose fiber, or a combination thereof, wherein the absorbent core comprises z-direction deformations;
wherein the absorbent pad exhibits a Conformability Force at 5 mm less than about 3500 N/m and greater than about 260 N/m, and an average dry caliper of about 2 mm to 7 mm.

2. The absorbent pad of claim 1, having a tapered, shape having a wider forward end region that tapers to a narrower rearward end region.

3. The absorbent pad of claim 1, wherein an outward-facing surface of the backsheet comprises an adhesive.

4. The absorbent pad of claim 1, wherein an outward-facing surface of the backsheet comprises a discontinuous layer of adhesive, where a total area of an adhesive-coated region(s) is less than 100% of the total surface area of the outward-facing surface of the backsheet.

5. The absorbent pad of claim 4, wherein the outward-facing surface of the backsheet comprises an array of separate lines of adhesive.

6. The absorbent pad of claim 1, wherein the deformations are discrete.

7. The absorbent pad of claim 1, wherein the deformations are plastic deformations.

8. The absorbent pad of claim 1, wherein the deformations are substantially continuous.

9. The absorbent pad of claim 1, wherein the deformations are formed by mechanically processing the absorbent core, wherein each deformation defines a region that has been stretched and plastically deformed.

10. The absorbent pad of claim 1, wherein the deformations are present at a numerical density of 3.0 to 9.0 deformations per cm², with a CD spacing of 2-15 mm, and an MD spacing of 2-15 mm.

11. The absorbent pad of claim 1, wherein the absorbent core comprises a first surface, a second surface, land areas, and deformations comprising protrusions extending outward from the second surface of the absorbent core, wherein the protrusions are surrounded by the land areas.

12. The absorbent pad of claim 11, wherein a majority of the protrusions comprise a base forming an opening, an opposed distal portion, and one or more side walls between the bases and the distal portions of the majority of the protrusions, and wherein the base, distal portion and the one or more side walls are formed by fibers such that the majority of the protrusions have only one opening at the base.

13. The absorbent pad of claim 11, wherein the majority of the protrusions comprises an inside void volume.

14. The absorbent pad of claim 1, wherein the topsheet is joined to the backsheet to form a perimeter seal.

15. The absorbent pad of claim 1, wherein the absorbent core structure is substantially free of super absorbent polymer (SAP).

16. The absorbent pad of claim 1, wherein the absorbent core comprises one or more layers of a combination of nonwoven web material and a web or batt of cellulose fiber.

17. The absorbent pad of claim 1 comprising an adhesive disposed at an interface between the topsheet and the absorbent core, an interface between the backsheet and the absorbent core, or both.

18. The absorbent pad of claim 1, configured as an incontinence insert.

19. An underpants garment in combination with the absorbent pad of claim 1.
